(19) United States
(12) Patent Application Publication
(10) Pub. No.: US 2005/0049516 A1
(43) Pub. Date: Mar. 3, 2005

(54) Methods, systems and computer program products for selectively initiating interventional therapy to reduce the risk of arrhythmia

(76) Inventor: Raymond E. Ideker, Birmingham, AL (US)

Correspondence Address: Laura M. Kelley;Myers Bigel Sibley & Sajovec, P.A., P. O. Box 37428, Raleigh, NC, 27627, US

(21) Appl. No.: 10/648,162

(22) Filed: Aug. 26, 2003

(51) Int. Cl. A61N 1/362; A61N 1/36

(52) U.S. Cl. 600/516; 607/9; 607/25; 607/4

(57) ABSTRACT

Electrical activity can be chronically detected in first and second cardiac regions in the subject. Discordant alternans in at least one component of the detected electrical activity can by identified. Interventional therapy can be initiated in the subject responsive to the identification of discordant alternans.

### FIELD OF THE INVENTION

The present invention relates to cardiac therapy, and more specifically to antiarrhythmic therapies.

### BACKGROUND OF THE INVENTION

Despite advances in antiarrhythmic therapies, cardiac arrhythmias remain a major health problem, causing about 300,000 sudden cardiac deaths annually in the United States (Weiss J N et al., Circulation (1999) 99:2819-2826). Cardiac arrhythmias can occur when the electrical waves which stimulate the heart meander erratically through the heart muscle, creating disordered and ineffective contraction. The primary focus of literature and research has been on detecting when cardiac arrhythmias occur and reducing the occurrence of arrhythmias with medical therapies or lifestyle changes. Medical therapies include drugs which can reduce the occurrence of arrhythmias and implantable devices which can detect the onset of arrhythmias and apply electrical pulses to the heart to stop arrhythmias.

### SUMMARY OF THE INVENTION

According to embodiments of the present invention, methods, systems, and computer program products for selectively initiating interventional therapy in a subject are provided. Electrical activity can be chronically detected in first and second cardiac regions in the subject. Discordant alternans in at least one component of the detected electrical activity can by identified. Interventional therapy can be initiated in the subject responsive to the identification of discordant alternans.

Identifying discordant alternans can be based on cycle-to-cycle variations in the detected electrical activity. In some embodiments, the component in which discordant alternans is detected includes a duration and/or amplitude of an STT segment. Initiating interventional therapy can be responsive to a change in the component from concordant to discordant alternans. The interventional therapy may reduce the risk of arrhythmia, including the risk of ventricular arrhythmia and/or atrial arrhythmia. For example, the interventional therapy may introduce a pacing routine, administer a shock, and/or administer a drug that reduces a risk of arrhythmia.

In some embodiments, the electrical activity comprises an ECG signal from external electrodes and/or an electrogram from internally implanted electrodes. The component can be the duration of a cardiac signal component, the amplitude of a cardiac signal component, and/ or the shape of a cardiac signal component.

As will further be appreciated by those of skill in the art, while described above primarily with reference to method aspects, the present invention may be embodied as methods, apparatus/systems and/or computer program products.

### BRIEF DESCRIPTION OF THE DRAWINGS

FIG. 1 is a block diagram of a device according to embodiments of the present invention;

FIG. 2 is a block diagram of operational circuitry according to embodiments of the present invention;

FIG. 3 is a block diagram of operational circuitry and/or computer program modules suitable for carrying out operations according to embodiments of the present invention;

FIG. 4 is a schematic illustration of an implantable apparatus with exemplary electrode placements according to embodiments of the present invention;

FIG. 5 is a flowchart illustrating operations that can be carried out according to embodiments of the present invention; and

FIG. 6 is a graph of cardiac cycles illustrating concordant and disconcordant alternans according to embodiments of the present invention.

### DETAILED DESCRIPTION OF EMBODIMENTS OF THE INVENTION

The present invention will now be described more fully hereinafter with reference to the accompanying figures, in which embodiments of the invention are shown. This invention may, however, be embodied in many different forms and should not be construed as limited to the embodiments set forth herein. Like numbers refer to like elements throughout. In the figures, certain regions, components, features or layers may be exaggerated for clarity. Broken lines where used indicate optional features, components or operations. It will be understood that when an element is referred to as being “coupled” or “connected” to another element, it can be directly coupled or connected to the other element or intervening elements may also be present. In contrast, when an element is referred to as being “directly coupled” or “directly connected” to another element, there are no intervening elements present.

The flowcharts and block diagrams of certain of the figures herein illustrate the architecture, functionality, and operation of possible implementations for predicting arrhythmias and/or selectively initiating interventional therapy according to the present invention. In this regard, each block in the flow charts or block diagrams represents a module, segment, or portion of code, which comprises one or more executable instructions for implementing the specified logical function(s). It should also be noted that in some alternative implementations, the functions noted in the blocks may occur out of the order noted in the figures. For example, two blocks shown in succession may in fact be executed substantially concurrently or the blocks may sometimes be executed in the reverse order, depending upon the functionality involved. In addition, some functions noted in the blocks may be combined or separated. While the present invention is illustrated in certain of the figures with reference to particular divisions of programs, functions and memories, the present invention should not be construed as limited to such logical divisions. Thus, the present invention should not be construed as limited to the configuration of operation as shown in the figures, but is intended to encompass any configuration capable of carrying out the operations described herein.

The present invention is intended primarily for use on human subjects, but may optionally be carried out on other mammalian subjects for veterinary purposes.

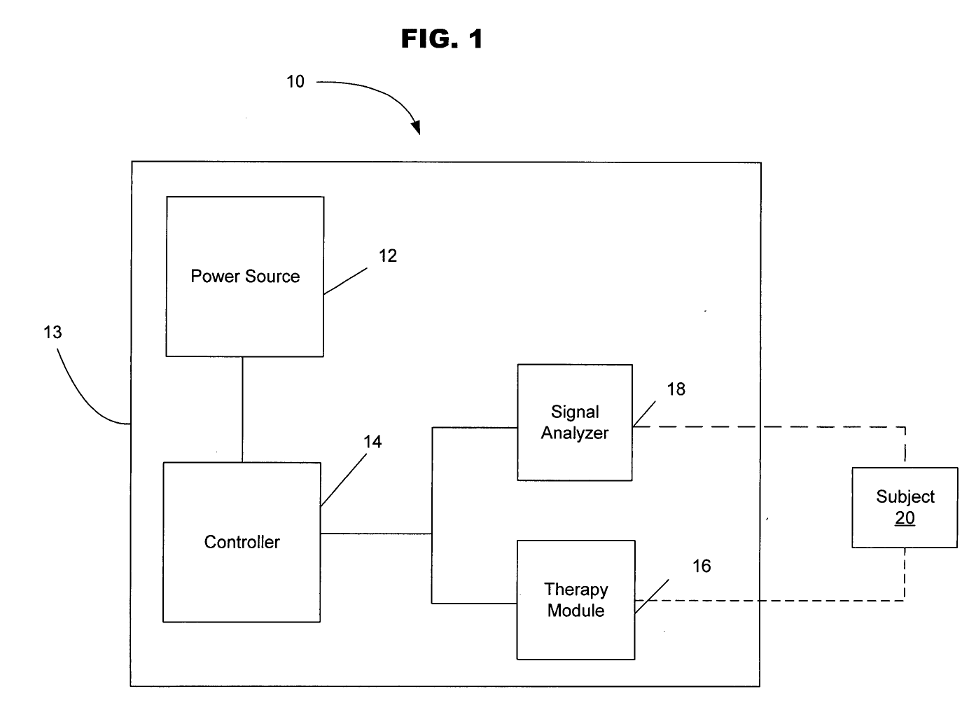

Referring to FIG. 1, an exemplary cardiac device 10 is shown. The device 10 includes a housing 13, a power source 12 held in the housing 13, and a controller 14 held in the housing 13 and operatively associated with the power source 12. A signal analyzer 18 is operatively associated with the controller 14 and receives a signal that represents electrical activity in the heart of a subject 20. The signal analyzer 18 analyzes a cardiac signal and determines if a therapy should be initiated and/or administered to the subject 20 by a therapy module 16.

Accordingly, electrical activity in the heart of a subject can be chronically detected and interventional therapy can be selectively administered. Chronic detection of electrical activity refers to the detection of electrical activity over an extended duration of time. The detection of electrical activity is not necessarily continuous and interruptions in detection may occur; however, in some embodiments, continuous detection of electrical activity may be provided. In some embodiments, electrical activity for successive cardiac cycles can be detected from a system chronically implanted in a subject.

Referring to FIG. 1, signals representing electrical activity in more than one cardiac region in the subject can be received by the signal analyzer 18. The signal analyzer 18 can identify discordant alternans in at least one component of the detected electrical activity, for example, by comparing a component of the signal received from two or more cardiac regions over cardiac cycle(s). Alternans is a change in the amplitude and/or morphology of a component of electrical activity in the heart, such as in an electrocardiogram (ECG), that occurs on an alternating basis, such as every-other-beat. Discordant alternans is alternans that occur on an alternating basis at different cardiac regions. According to embodiments of the present invention, interventional therapy can be initiated in the subject responsive to the identification of discordant alternans, for example, by detecting a relative change in the component at the cardiac regions either between different sensing locations in the same cycle or the same location over different cycles. For example, if the signal analyzer 18 detects a relative change in a component of the electrical signal at the cardiac regions, then the therapy module 16 can deliver a therapeutic treatment to reduce a risk of arrhythmia. In some embodiments, the relative change can be a millivolt change or smaller. Further examples of detection methods can be found, for example, in U.S. Pat. Nos. 4,802,491 and 5,148,812, the disclosures of which are incorporated by reference in their entireties.

As an overview of a cardiac signal and examples of cardiac components, the driving force for the flow of blood in the heart comes from the active contraction of the cardiac muscle. An electrical signal causes this contraction of the heart. The electrical signals described herein can be detected as an ECG signal from external electrodes situated on the surface of the patient and/or from internally implanted electrodes. Electrical signal components from external and/or internal electrodes can be used to detect alternans. The cardiac contraction is triggered by electrical impulses traveling in a wave propagation pattern, which begins at the cells of the sinoatrial node and the surrounding atrial myocardial fibers, and then traveling into the atria and subsequently passing through the atrioventricular node and, after a slight delay, into the ventricles.

The beginning of a cardiac cycle is initiated by a P wave, which is normally a small positive wave in the body surface electrocardiogram. The P wave induces depolarization of the atria of the heart. The P wave is followed by a cardiac cycle portion which is substantially constant with a time constant on the order of 120 milliseconds (“ms”).

The “QRS complex” of the cardiac cycle occurs after the substantially constant portion. The dominating feature of the QRS complex is the R wave which is a rapid positive deflection. The R wave generally has an amplitude greater than any other wave of the cardiac cycle, and has a spiked shape of relatively short duration with a sharp rise, a peak amplitude, and a sharp decline. The QRS complex is the depolarization of the ventricles and therefore, the term “ventricle activations” denotes a QRS complex of the cardiac cycle. The QRS complex is completed by the S wave, which is typically a small deflection that returns the cardiac signal to baseline.

Following the S wave, the T wave occurs after a delay of about 250 ms. The T wave is relatively long in duration (e.g., about 150 ms). The cardiac cycle between the S wave and the beginning of the T wave is commonly referred to as the ST segment. The STT segment refers to the cardiac cycle between the S wave and the end of the T wave. The T wave is a sensitive part of the cardiac cycle, during which an electrical stimulus, such as an atrial defibrillation shock, is to be avoided, in order to reduce the possibility of induced (and often fatal) ventricular fibrillation. The next cardiac cycle begins with the next P wave. The typical duration of a complete cardiac cycle is on the order of about 800 ms.

In some embodiments, an electrogram recorded from an electrode on or in the heart can be used to detect alternans. Such an electrogram can include an activation complex and a repolarization complex. The activation complex can be referred to as a QRS or RS complex and may be recognized as a rapid downslope in a recording from a unipolar electrode and as a spike in a recording from a bipolar electrode. The repolarization complex may be referred to as a T wave and may be more prominent in a unipolar than in a bipolar recording. The activation recovery interval (ARI) is a measurement proportional to the refractory period and to the action potential duration of the tissue around the electrode. The ARI can be calculated as the time from the fastest downstroke of the activation complex of the unipolar electrogram to the fastest upstroke of the T wave of the unipolar electrogram.

Accordingly, any cardiac signal component (e.g., STT segment, R wave, T wave, ARI, QRS complex, etc.) can be identified to detect alterants. Moreover, various characteristics of cardiac signal components can be used to detect alterants, including the duration of a cardiac signal component, the amplitude of a cardiac signal component, the shape of a cardiac signal component, and the like. Alternans can also include alternating patterns having periods of varying lengths. For example, a characteristic of a component in the cardiac signal used to identify discordant alternans can repeat ever other beat, every fourth beat, every sixth beat and so on.

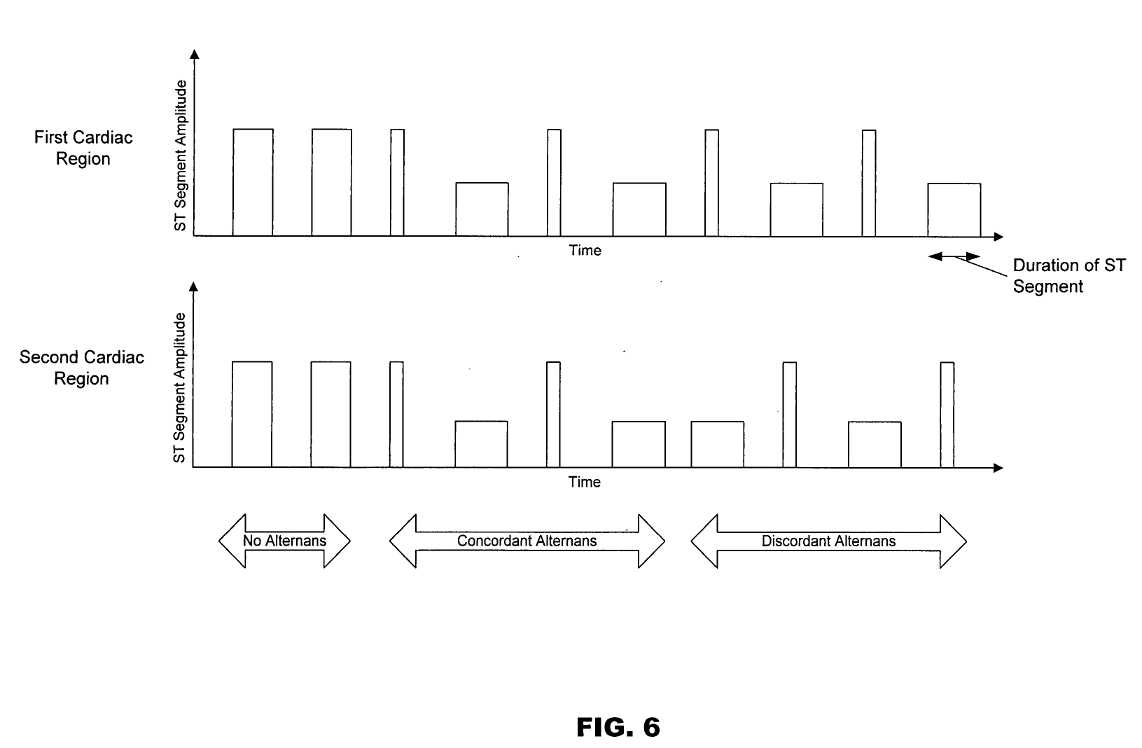

An exemplary graph of STT segment duration and amplitude illustrating a general pattern including cycle-to-cycle STT segments having no alterants, concordant alternans, and discordant alterants is shown in FIG. 6. In concordant alternans, different portions of the myocardial region exhibiting alternans are in phase with one another. That is, electrical signals detected at different points in the myocardial region each exhibit the same alternating pattern from beat to beat if a patient is experiencing concordant alternans. For example, as shown in FIG. 6, a taller (ie., greater amplitude) or longer duration STT segment can alternate beat to beat with a smaller (i.e., smaller amplitude) or shorter duration STT segment simultaneously at different myocardial spatial regions. However, in the case of discordant alternans, different portions of the myocardial region can be out of phase with one another. For example, one portion of the myocardium can exhibit a taller or longer STT segment while another portion exhibits a smaller or shorter STT segment during the same beat. In the next beat, the relative amplitude or duration of the STT segment is reversed. That is, the portion of the myocardium exhibiting the longer STT segment during the previous beat next exhibits a shorter STT segment, and the portion that exhibited the shorter STT segment during the first beat exhibits a longer STT segment in the second beat. Various other components of a cardiac signal can be used to detect discordant alternans.

Without wishing to be bound by any particular theory, it is believed that changes in cardiac signal components (e.g., STT segment duration and/or amplitude) of a cardiac cycle over time in which comparisons between different cardiac locations can diverge, such as in the onset of discordant alternans, may indicate a heightened risk of arrhythmia. Accordingly, the risk of arrhythmia may be predicted and/or reduced with interventional therapy prior to the onset of arrhythmia. Embodiments of the present invention may be applied to various forms of cardiac tachyarrhythmias, including atrial and ventricular fibrillation, with defibrillation (including cardioversion) shocks or pulses and/or pacing routines. Examples include the prevention and/or treatment of polymorphic ventricular tachycardia, monomorphic ventricular tachycardia, ventricular fibrillation, atrial flutters, and atrial fibrillation.

As shown in FIG. 1, the therapy module 16 is configured to deliver one or more therapeutic treatments to reduce a risk of arrhythmia responsive to a relative change in a cardiac signal component as determined by the signal analyzer 18. Any suitable interventional therapy may be used, including therapies that reduce the risk of atrial and/or ventricular arrhythmia, such as administering a pacing routine, an electrical shock (such as a defibrillation shock), or a drug. Examples of drugs that can be used include calcium channel blockers, calmodulin blockers, calmodulin kinase inhibitors, beta blockers and antiarrhythmic drugs. Examples of drug delivery systems are provided in co-assigned application Ser. No. 10/071,269, entitled Methods and Devices for Treating Arrhythmias Using Defibrillation Shocks, filed Feb. 8, 2002, the disclosure of which is incorporated by reference in its entirety. The therapy module 16 can automatically deliver a therapeutic treatment to reduce the risk of arrhythmia, for example, by automatically delivering a pacing routine, a defibrillation shock and/or a therapeutic drug. The treatment can also be delivered manually. For example, in some embodiments, the therapy module 16 notifies a user, such as a health care professional or the patient, that a therapeutic treatment should be administered to the patient.

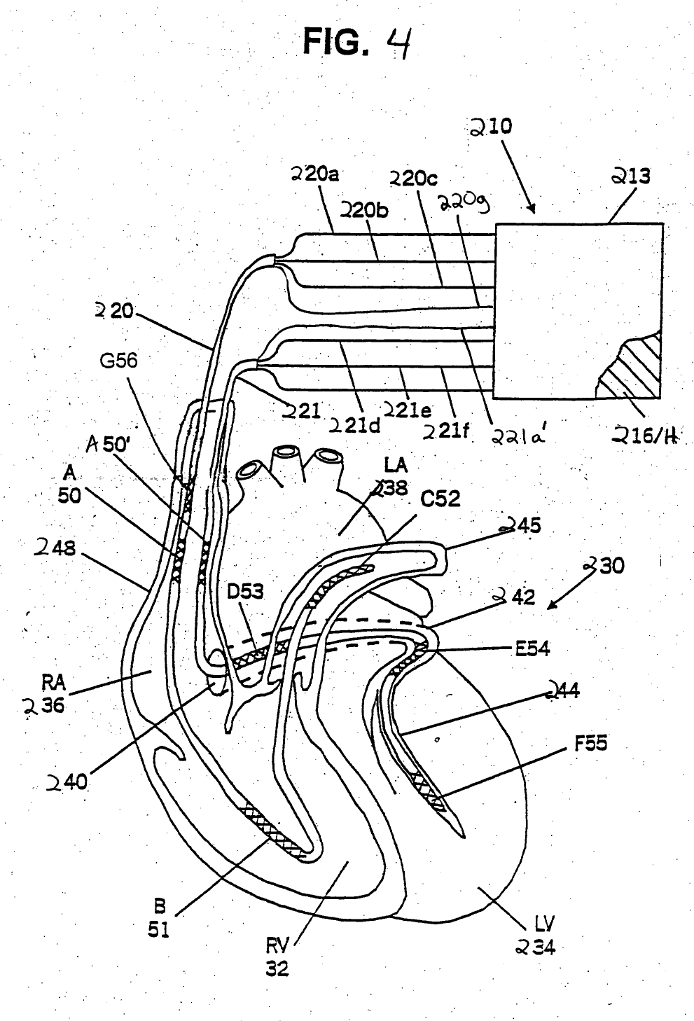

Various pacing routines, including pacing routines known to those of skill in the art, can be used. For example, the pacing routines can include one or more pulses from electrodes in various cardiac locations, including electrodes that can also be used to detect alternans and/or the electrode configuration shown in FIG. 4. Pacing routines can be administered as a single pulse or a series of pulses from one or more electrodes. Pacing routines can also be administered simultaneously from multiple electrodes. In some embodiments, the pacing routine can be timed based on the spatial and/or temporal pattern of the detected alternans. For example, a pacing routine can be timed to stimulate a cardiac region coinciding with the detection of a shorter STT segment in the same region. The pacing routine could also be timed to stimulate a cardiac region during or after a beat exhibiting a shorter or longer STT segment.

The device 10 can be an external or internal device. Accordingly, the signal analyzer module 18 can receive electrical activity of the heart from internal electrodes by an implantable anti-arrhythmic device or from external electrodes by an external anti-arrhythmic device. Moreover, the therapy module 16 can administer a pacing routine or defibrillation shock from internal or external electrodes. In the case of drug therapies, the therapy module 16 can administer a drug automatically from an internally implantable drug delivery system as described, for example, U.S. application Ser. No. 10/071,269. Interventional therapies can be administered alone or in combination with other therapies. For example, a pacing routine and/or defibrillation shock can be administered before, at the same time, or after a drug is delivered.

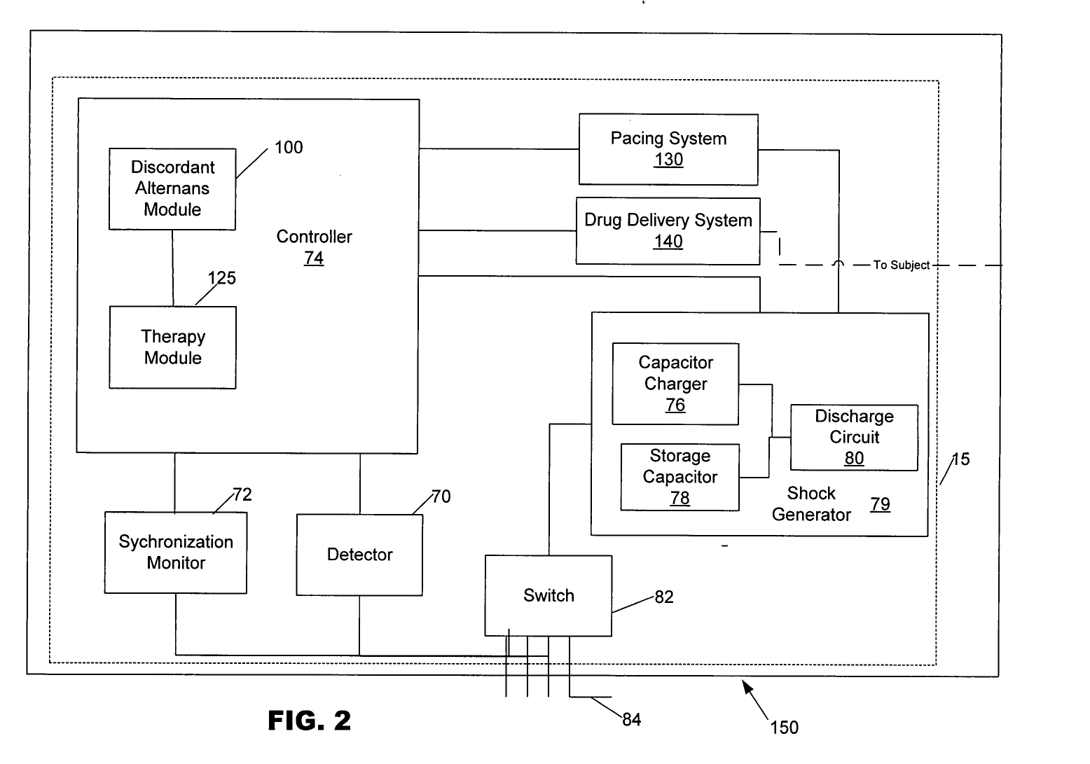

FIG. 2 illustrates a device 150 according to further embodiments of the invention, which contains an electronic circuit 15. The electrical circuit 15 can include circuitry that can sense or detect electrical signals in a cardiac region (e.g., from electrodes positioned to sense the electrical signals in the cardiac region), analyze the electrical signals, and/or control the delivery of appropriate therapies, such as shocks to the cardiac region (such as defibrillation shocks and/or pacing routines) and/or drug delivery.

As illustrated in FIG. 2, the electrical circuit 15 includes leads 84 that are electrically connected to external and/or internal electrodes (FIG. 4) placed in electrical contact with a heart, a switch 82 for controlling signals to and from the leads 84, an atrial and/or ventricular detector 70 that receives and analyzes cardiac signals that are received by the leads 84, and a cardiac cycle monitor or “synchronization monitor 72”) for providing synchronization information to a controller 74. The controller 74 controls a shock generator 79, which includes a capacitor charging circuit 76 that charges the storage capacitor 78 to a predetermined voltage, typically from a power source such as a battery source (not shown). The controller 74 can direct a discharge circuit 80 to discharge an electrical current from the shock generator 79 to the switch 82 into leads 84. Accordingly, leads 84 can provide electrical sensing and/or shocking functionality. The controller 74 also includes a discordant alternans module 100 and a therapy module 125. The controller 74 also controls a drug delivery system 140 for delivering a drug and a pacing system 130 for monitoring cardiac cycles from the electrical signals from the heart sensed by the electrodes and for providing a pacing routine.

Still referring to FIG. 2, generally described in operation, upon receiving a signal from the leads 84 and the detector 70, the discordant alternans module 100 of the controller 74 analyzes the signal. The signal can represent electrical activity in two or more cardiac regions. The discordant alterants module 100 compares a segment in a cardiac cycle represented by the electrical activity at the cardiac regions. The therapy module 125 initiates and/or controls the administration of an interventional therapy responsive to a relative change in the component at the two cardiac regions. The relative change in the component can be monitored over a plurality of cardiac cycles to detect cycle-to-cycle variations that can indicate that therapy is needed. The administered therapy can be a defibrillation shock, a pacing routine, and/or a delivery of a drug. For example, in some embodiments, the therapy module 125 can direct a drug to be delivered from the drug delivery system 140, a pacing routine to be delivered from the pacing system 130, and/or a shock to be delivered from the shock generator 79. Moreover, the pacing system 130 can communicate with the shock generator 79 to control a pacing routine delivered to leads 84 via the switch 82.

For example, the therapy module 125 can signal the shock generator 79 to generate a defibrillation shock and/or pacing routine having particular characteristics. The capacitor charging circuit 76 of the shock generator 79 charges the storage capacitor 78 to a predetermined voltage. The storage capacitor 78 can be 20 to 400 microfarads in size, and may be a single capacitor or a capacitor network (e.g., separate pulses can be driven by the same or different capacitors). The discharge of the capacitor 78 may be controlled by the controller 74 and/or a discharge circuit 80. The controller 74, based on information from the synchronization monitor 72, can direct the shock to be relayed to either the discharge circuit 80 for further processing (i e., to further shape the waveform signal, time the. pulse or pulses, etc.) or directly to an output lead or to a switch, such as switch 82. The controller 74 may also control the desired or proper selection of predetermined defibrillation electrode pair(s), where multiple defibrillation electrodes are used, to direct the switch 82 to electrically activate a desired electrode pair to align the predetermined electric shock pulse pathway through which the shock pulse is provided. As an alternative, the therapy module 125 can provide an alert to administer the shock profiles and/or pulse sequences. For example the therapy module can provide a local or remote audible and/or visual alert to a patient or to a health care professional.

In some embodiments, the pulse generator includes a single capacitor 78, and the controller 74 includes a switch (e.g., a crosspoint switch) operatively associated with that capacitor. Various shock profiles and/or shock sequences can be used. For example, the controller 74 may be configured to provide a shock profile consisting of a biphasic pulse (i.e., a first phase of a pulse of a predetermined polarity followed by a second phase of a pulse of reversed polarity). Single pulses and/or sequences of pulses, including monophasic, biphasic, and/or triphasic pulses may also be used. Various shock profiles may be used having various properties including waveform, duration, polarity, shape, periodicity, energy, voltage, etc. Exemplary shock profiles are described in U.S. Pat. No. 6,327,500 to Cooper et al., U.S. Pat. No. 5,978,705 to KenKnight et al. U.S. patent application Ser. No. 10/012,115 (Publication No. 02 0161407) filed Nov. 13, 2001, the contents of which are hereby incorporated by reference as if recited in full herein.

The controller 74 can deliver a preselected electrical pulse to predetermined electrode pairs through a switch 82. The shock generator 79 (including a capacitor charger 76, capacitor 78, and discharge circuit 80), controller 74, and switch 82 thus work in concert to produce and deliver a pulse having a particular shock profile. Therefore, it will be appreciated that in operation, in response to an input from the detector 70, the discordant alternans module 100 and/or the therapy module 125, the controller 74 controls the pulse or shock generator 79 to synchronize the delivery of the timed pulse output to the proper electrode pair in accordance with the cardiac cycle information received from the synchronization monitor 72 and the specific electrode configuration employed by or selected by the device. Further, when employing a biphasic waveform, it will be appreciated by those of skill in the art that the pulse or shock generator 79 can also include a crosspoint switch to switch the polarity of the electrode pair for delivery of the second (inverted or negative) waveform phase. The electronic package may also include a receiver/transmitter coupled to the internal controller 74 for communicating with an external controller. Thus, the pulse regimen could be altered by external input to the controller to alter, for example, the waveform, the voltage, the electrode coupling, or even to retrieve monitoring data received and stored in memory about the number of atrial fibrillation episodes and the effectiveness of the shock level.

In some embodiments, the switch 82 is programmable (e.g., by remote control such as by a radio signal) to alter the coupling of the pulse generator to the atrial defibrillation electrodes. This feature may be particularly suitable when multiple electrodes are implanted so that the electrode pairs that deliver the shocks may be changed to optimize the technique for a particular patient.

The electrical circuit 15 can include one or more amplifiers (not shown) for amplifying the sensed cardiac signals. Defibrillation and/or pacing electrodes may be configured to sense cardiac cycles from electrical signals from the heart, or may have smaller sensing electrodes placed adjacent thereto and thereby provide input to the electronics package as well as provide a predetermined stimulation shock output to predetermined cardiac areas as directed by the controller 74. The synchronization monitor 72 can provide additional assurance that defibrillation shock pulses are not delivered during sensitive portions of the cardiac cycle so as to reduce the possibility of inducing ventricular fibrillation.

The present invention should not be construed as limited to the configuration of FIG. 2, which is intended to encompass any configuration capable of carrying out the operations described herein, including implantable and external configurations.

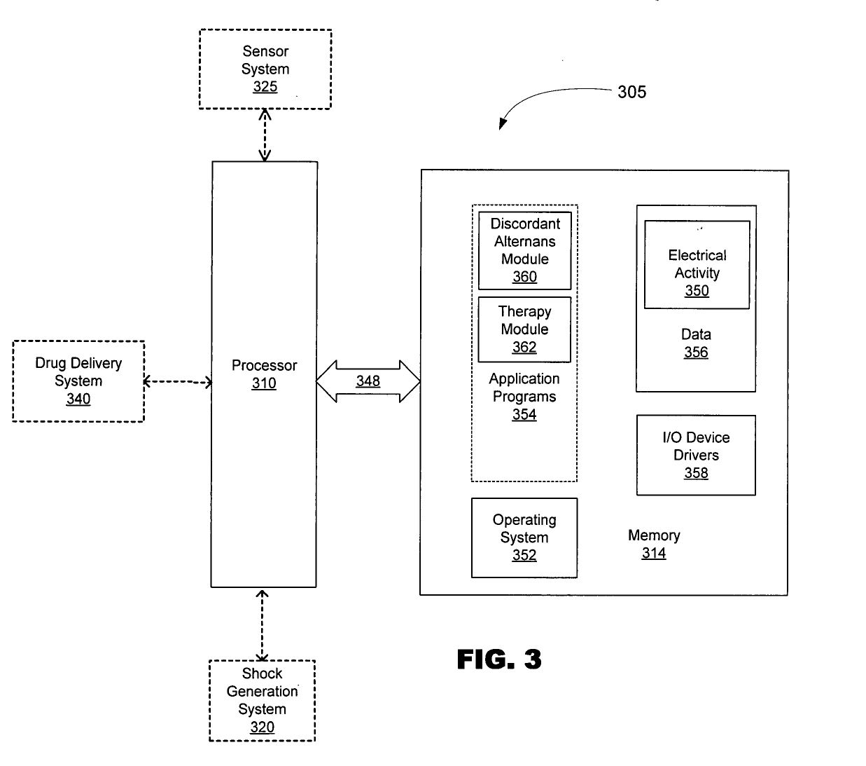

FIG. 3 is a block diagram of exemplary embodiments of data processing systems that illustrates systems, methods, and computer program products in accordance with embodiments of the present invention. The data processing system 305 includes a processor 310 that can send and receive information to or from a sensing system 325 and a shock generation system 320 and/or a drug delivery system 340. The data processing system 305 may be implemented externally or internally with respect to the patient. The shock generation system 320 and/or the sensing system 325 may be implanted in the patient or be implemented externally.

The processor 310 communicates with the memory 314 via an address/data bus 348. The processor 310 can be any commercially available or custom microprocessor. The memory 314 is representative of the overall hierarchy of memory devices containing the software and data used to implement the functionality of the data processing system 305. The memory 314 can include, but is not limited to, the following types of devices: cache, ROM, PROM, EPROM, EEPROM, flash memory, SRAM, and DRAM.

As shown in FIG. 3, the memory 314 may include several categories of software and data used in the data processing system 305: an operating system 352; application programs 354; input/output (I/O) device drivers 358; a discordant alternans module 360, a therapy module 362 and data 356. The data 356 may include electrical activity data 350, such as an ECG signal or electrogram, which may be obtained from a electrical sensor for detecting electrical activity in the cardiac region, for example, from the sensing system 325.

As will be appreciated by those of skill in the art, the operating system 352 may be any operating system suitable for use with a data processing system, such as OS/2, AIX, OS/390 or System390 from International Business Machines Corporation, Armonk, N.Y., Windows CE, Windows NT, Windows95, Windows98 or Windows2000 from Microsoft Corporation, Redmond, Wash., Unix or Linux or FreeBSD, Palm OS from Palm, Inc., Mac OS from Apple Computer, or proprietary operating systems. The I/O device drivers 358 typically include software routines accessed through the operating system 352 by the application programs 354 to communicate with devices such as I/O data port(s), data storage 356 and certain memory 314 components and/or the shock generation system 320, sensing system 325 and/or drug delivery system 340. The application programs 354 are illustrative of the programs that implement the various features of the data processing system 305 and preferably include at least one application which supports operations according to embodiments of the present invention. Finally, the data 356 represents the static and dynamic data used by the application programs 354, the operating system 352, the I/O device drivers 358, and other software programs that may reside in the memory 314.

While the present invention is illustrated, for example, with reference to the discordant alternans module 360 and the therapy module 362 being an application program in FIG. 3, as will be appreciated by those of skill in the art, other configurations may also be utilized while still benefiting from the teachings of the present invention. For example, the discordant alternans module 360 and/or the therapy module 362 may also be incorporated into the operating system 352, the I/O device drivers 358 or other such logical division of the data processing system 305. Thus, the present invention should not be construed as limited to the configuration of FIG. 3, which is intended to encompass any configuration capable of carrying out the operations described herein.

The I/O data port can be used to transfer information between the data processing system 305 and the shock generation system 320, sensing system 325, or another computer system or a network (e.g., the Internet) or to other devices controlled by the processor. These components may be conventional components such as those used in many conventional data processing systems that may be configured in accordance with the present invention to operate as described herein.

Accordingly, the sensing system 325 can send an electrical signal, such as an ECG or electrogram signal, to the processor 310. The electrical signal can be stored as electrical activity data 350. The discordant alternans 360 can compare electrical signals at different positions in the cardiac region to determine relative changes, such as variations in a single cycle between the positions, discordant alternans and other cycle-to-cycle changes. In response to a detected relative change, the therapy module 362 can initiate a therapy. For example, the therapy module 362 can instruct the shock generation system 320 to administer a shock, such as a defibrillation shock and/or pacing routine. The therapy module 362 can instruct the drug delivery system 340 to deliver a therapeutic drug. In some embodiments, the therapy module 362 alerts a user, such as a health care professional, that interventional therapy should be administered. The therapy module 362 can also select one of several therapies based on the particular relative change detected by the discordant alternans 360.

In some embodiments, various functionalities discussed herein can be implemented in an internally implantable system as shown in FIG. 4, although as noted previous, external systems can also be used. Anatomically, the heart includes a fibrous skeleton, valves, the trunks of the aorta, the pulmonary artery, and the muscle masses of the cardiac chambers (ie., right and left atria and right and left ventricles). The schematically illustrated portions of the heart 230 illustrated in FIG. 4 includes the right ventricle “RV” 232, the left ventricle “LV” 234, the right atrium “RA” 36, the left atrium “LA” 238, the superior vena cava 248, the coronary sinus “CS” 242, the great cardiac vein 244, the left pulmonary artery 245, and the coronary sinus ostium or “os” 240.

Referring to FIG. 4, the device 210 can include an implantable housing 213 that contains a hermetically sealed electronic circuit, such as the circuit 15 as shown in FIG. 2. The device 210 can be configured detect electrical activity and/or to administer defibrillation and/or pacing routines according to embodiments of the present invention. The housing 213 can include an electrode comprising an active external portion 216/H of the housing, with the housing 213 preferably implanted in the left thoracic region of the patient (e.g., subcutaneously, in the left pectoral region) in accordance with known techniques as described in G. Bardy, U.S. Pat. No. 5,292,338. As shown, the system can include a first catheter 220 and a second catheter 221, both of which are insertable into the heart (typically through the superior or inferior vena cava) without the need for surgical incision into the heart. The term “catheter” as used herein includes “stylet” and “lead” interchangeably. Each of the catheters 220, 221 contains electrode leads wires 220a, 220b, 220c, 221a′, 221d, 221e, 221f, and 220g respectively, with the small case letter designation corresponding to the large-case letter designation for the defibrillation electrode to which each lead wire is electrically connected.

As illustrated in FIG. 4, the catheter 220 includes electrodes A50 and G56 that reside in the right atrium “RA” (the term “right atrium” herein including the superior vena cava and innominate vein), an electrode B51 positioned in the right ventricle (preferably in the right ventricular apex), and an electrode C52 positioned within the left pulmonary artery (the term “left pulmonary artery” herein includes the main pulmonary artery and the right ventricular outflow tract).

The second catheter lead 221 includes, from proximal to distal, an electrode A50′ in the right atrium; an electrode D53 positioned in the proximal coronary sinus, adjacent the coronary sinus ostium or “OS” 240; an electrode E55 positioned in the distal coronary sinus (preferably as far distal in the coronary sinus as possible) (the term “distal coronary sinus” herein includes the great cardiac vein); and an electrode F56 at or adjacent the tip of the catheter in a coronary vein on the surface (preferably the posterolateral surface) of the left ventricle (e.g., in the lateral-apical left ventricular free wall). The position of electrode F56 may be achieved by first engaging the coronary sinus with a guiding catheter through which a conventional guidewire is passed. The tip of the torqueable guidewire is advanced under fluoroscopic guidance to the desired location. The lead 221 on which electrode F56 is mounted passes over the guidewire to the proper location. The guidewire is withdrawn and electrode F56 is incorporated into the defibrillation lead system.

The active external portion of the housing 216 serves as an optional electrode H, which may be used for either atrial or ventricular defibrillation.

As illustrated in FIG. 4, any or all of the electrodes can sense discordant alternan electrical signals and transmit the signals to the device 210. The electrodes shown in FIG. 4 can also be configured to provide a defibrillation pulse, pacing routine and/or cardiac resynchronization therapy (CRT), and in some embodiments, an electrode can be used for providing both sensing and pulsing functionality. For example, in some embodiments, two electrodes configured for CRT can be used to detect alternans and/or deliver pulses, including defibrillation pulses, pacing routines, and/or CRT pulses. The two electrodes configured for CRT can be situated in the right and left ventricles according to known techniques. Moreover, it will be appreciated by those of skill in the art that various electrode configurations, including additional sensing and/or pulsing electrode(s) in alternative cardiac areas, can be used. Additional sensing electrodes may also be placed near defibrillation electrodes. In some embodiments, sensing electrodes can be used to provide sensing signals to sensor input lines to a detector in the device 210. The sensing input can be used to compare cardiac signal components in a cardiac cycle, for example, using a discordant alternans module and/or signal analyzer as described herein. The electrodes can also be used to administer interventional therapy, such as a defibrillation pulse and/or pacing routine, responsive to a relative change in cardiac signal components in different cardiac regions.

Numerous configurations of capacitor and control circuitry may be employed as described herein. Additional features can also be added to the device 210 including, for example, safety features such as noise suppression or multiple wave monitoring devices (R and T), verification checking to reduce false positive, precardioversion warning, programmed delayed intervention, bipolar configured sensing electrodes, intermittently activated defibrillation detector to reduce energy drain, a switching unit to minimize lines from the pulse generator, etc.

Those skilled in the art will appreciate that various electrode combinations are possible for both atrial and ventricular defibrillation and/or pacing by employing the “active can” electrode H, as discussed herein. In addition, multiple electrodes can be electrically coupled or “tied” together to form a single pole. For example, a shock can be delivered from either the RV or LV as one pole to the PA and OS tied together as the other pole.

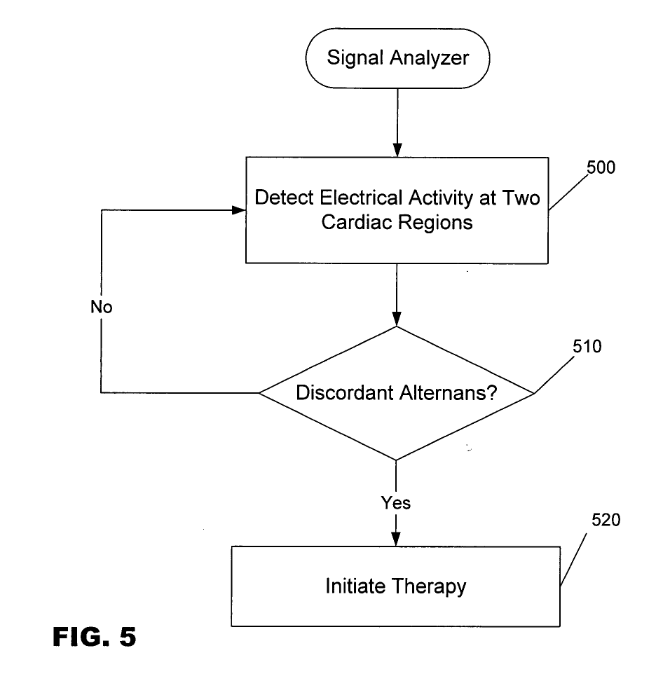

Operations according to embodiments of the present invention are shown in FIG. 5. A signal analyzer detects electrical activity at two cardiac regions in the subject at Block 500. If discordant alternans are identified at Block 510, then the signal analyzer triggers the administration of interventional therapy at Block 520.

Systems as described above may be implanted in a patient by conventional surgical techniques, or techniques readily apparent to skilled surgeons in light of the disclosure provided herein, to provide an implanted defibrillation or cardioversion system. Embodiments may include surface mounted, internally implanted, or external components or a combination thereof.

Embodiments of the present invention are described herein with reference to “defibrillation” electrodes, “defibrillation” shocks, and the like. It should be understood that “defibrillation” electrodes and shocks include electrodes and shocks that reduce the risk of the occurrence of fibrillation as well as electrodes and shocks that result in actual defibrillation of a fibrillating heart. Accordingly, a defibrillation shock from a defibrillation electrode can be delivered without actual fibrillation being present.

Although the system has been primarily described above as an implantable system, it will be appreciated by those of ordinary skill in the art that the invention could also be incorporated into an external system which employs catheters to position the electrodes within a patient's heart or other desired configuration.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein.

1. A method for selectively initiating interventional therapy in a subject, comprising:
chronically detecting electrical activity in first and second cardiac regions in the subject;
identifying discordant alternans in at least one component of the detected electrical activity; and
initiating interventional therapy in the subject responsive to the identification of discordant alternans.

2. The method of claim 1, wherein the component comprises a duration, shape and/or amplitude of an STT segment.

3. The method of claim 1, wherein the component comprises a duration, shape and/or amplitude of a T wave.

4. The method of claim 1, wherein the component comprises a duration, shape and/or amplitude of an activation recovery interval (ARI).

5. The method of claim 1, wherein the identifying discordant alternans is based on cycle-to-cycle variations in the detected electrical activity.

6. The method of claim 1, wherein initiating interventional therapy is responsive to a change in the component from concordant to discordant alternans.

7. The method of claim 1, wherein the interventional therapy reduces a risk of arrhythmia.

8. The method of claim 1, wherein the interventional therapy reduces a risk of ventricular arrhythmia.

9. The method of claim 1, wherein the interventional therapy reduces a risk of atrial arrhythmia.

10. The method of claim 1, wherein the interventional therapy comprises introducing a pacing routine.

11. The method of claim 1, wherein the interventional therapy comprises administering a shock.

12. The method of claim 1, wherein the interventional therapy comprises administering a drug that reduces a risk of arrhythmia.

13. The method of claim 1, wherein the electrical activity comprises an ECG signal from external electrodes.

14. The method of claim 1, wherein the electrical activity comprises an electrogram from internally implanted electrodes.

15. The method of claim 1, wherein the component includes a duration of a cardiac signal component.

16. The method of claim 1, wherein the component includes an amplitude of a cardiac signal component.

17. The method of claim 1, wherein the component includes a shape of a cardiac signal component.

18. A system for selectively initiating interventional therapy in a subject, comprising:
a plurality of electrodes configured and sized to chronically detect electrical activity in first and second cardiac regions;
a discordant alternans monitor operably associated with the electrodes, the discordant alternans monitor configured to identify discordant alternans in at least one component of the detected electrical activity; and to initiate interventional therapy in the subject responsive to the identification of discordant alternans.

19. The system of claim 18, wherein the electrodes are configured to be internally implantable in the subject.

20. The system of claim 18, wherein the electrodes are configured to reside external on the subject.

21. The system of claim 18, further comprising a drug delivery system operably associated with the discordant alternans monitor, wherein the discordant alternans monitor is further configured to initiate interventional therapy by controlling the drug delivery system.

22. The system of claim 18, wherein the electrodes are further configured to deliver a pulse to the respective cardiac regions, wherein the discordant alternans monitor is further configured to initiate interventional therapy by controlling the pulse to the electrodes.

23. The system of claim 22, wherein the pulse comprises a pacing routine.

24. The system of claim 22, wherein the pulse comprises a defibrillation pulse.

25. The system of claim 18, wherein the component comprises a duration, shape and/or amplitude of an STT segment.

26. The system of claim 18, wherein the component comprises a duration, shape and/or amplitude of a T wave.

27. The system of claim 18, wherein the component comprises a duration, shape and/or amplitude of an activation recovery interval (ARI).

28. The system of claim 18, wherein the discordant alternans monitor is configured to identify discordant alternans based on cycle-to-cycle variations in the detected electrical activity.

29. The system of claim 18, wherein the discordant alternans monitor is configured to initiate interventional therapy responsive to a relative change in a component by detecting a change from concordant to discordant alternans.

30. The system of claim 18, wherein the interventional therapy reduces a risk of arrhythmia.

31. The system of claim 18, wherein the interventional therapy reduces a risk of ventricular arrhythmia.

32. The system of claim 18, wherein the interventional therapy reduces a risk of atrial arrhythmia.

33. The system of claim 18, wherein the electrical activity comprises an ECG signal from external electrodes.

34. The system of claim 18, wherein the electrical activity comprises an electrogram from internally implanted electrodes.

35. The system of claim 18, wherein the component includes a duration of a cardiac signal component.

36. The system of claim 18, wherein the component includes an amplitude of a cardiac signal component.

37. The system of claim 18, wherein the component includes a shape of a cardiac signal component.

38. A computer program product for selectively initiating interventional therapy in a subject, the computer program product comprising:
a computer readable storage medium having computer readable program code embodied in said medium, said computer-readable program code comprising:
computer readable program code configured to chronically detect electrical activity in first and second cardiac regions in the subject;
computer readable program code configured to identify discordant alternans in at least one component of the detected electrical activity; and
computer readable program code configured to initiate interventional therapy in the subject responsive to the identification of discordant alternans.

39. The computer program product of claim 38, wherein the component comprises a duration, shape and/or amplitude of an STT segment.

40. The computer program product of claim 38, wherein the component comprises a duration, shape, and/or amplitude of a T wave.

41. The computer program product of claim 38, wherein the component comprises a duration, shape and/or amplitude of an activation recovery interval (ARI).

42. The computer program product of claim 38, wherein the computer readable program code configured to identify discordant alternans further comprises computer readable program code configured to identify discordant alternans based on cycle-to-cycle variations in the detected electrical activity.

43. The computer program product of claim 38, wherein the computer readable program code configured to initiate interventional therapy further comprises computer readable program code configured to initiate interventional therapy responsive to a change in the component from concordant to discordant alternans.

44. The computer program product of claim 38, wherein the interventional therapy reduces a risk of arrhythmia.

45. The computer program product of claim 38, wherein the interventional therapy reduces a risk of ventricular arrhythmia.

46. The computer program product of claim 38, wherein the interventional therapy reduces a risk of atrial arrhythmia.

47. The computer program product of claim 38, wherein the computer program code configured to initiate interventional therapy further comprises computer program code configured to introduce a pacing routine.

48. The computer program product of claim 38, wherein the computer program code configured to initiate interventional therapy further comprises computer program code configured to control the administration of a shock.

49. The computer program product of claim 38, wherein the computer program code configured to initiate interventional therapy further comprises computer program code configured to initiate the administration of a drug that reduces a risk of arrhythmia.

50. The computer program product of claim 38, wherein the electrical activity comprises an ECG signal from external electrodes.

51. The computer program product of claim 38, wherein the electrical activity comprises an electrogram from internally implanted electrodes.

52. The computer program product of claim 38, wherein the component comprises a duration of a cardiac signal component

53. The computer program product of claim 38, wherein the component comprises an amplitude of a cardiac signal component.

54. The computer program product of claim 38, wherein the component comprises a shape of a cardiac signal component.
